(19) United States
(12) Patent Application Publication
(10) Pub. No.: US 2009/0123519 A1
(43) Pub. Date: May 14, 2009

(54) Swellable hydrogel matrix and methods

(75) Inventors: Emily R. Rolfes, Eden Priarie, MN (US); Stephen J. Chudzik, St. Paul, MN (US); Pamela J. Reed, St. Paul, MN (US)

Correspondence Address: Kagan Binder, PLLC, 221 Main Street North, Suite 200, Stillwater, MN, 55082, US

(73) Assignee: SurModics, Inc.

(21) Appl. No.: 12/291,622

(22) Filed: Nov. 12, 2008

Related U.S. Application Data
(60) Provisional application No. 61/002,823, filed on Nov. 12, 2007.

(51) Int. Cl. A61F 2/02; A61K 47/32

(52) U.S. Cl. 424/423; 514/772.3

(57) ABSTRACT

The invention provides biocompatible polymeric hydrogel matrices having excellent durability and swellability. The matrices are formed from a macromer and photo-polymer combination. The matrices can be used in association with a medical device or alone. In some methods the polymeric matrix is placed or formed at a target site in which the matrix swells and occludes the target area.

### CROSS-REFERENCE TO RELATED APPLICATION

This application claims the benefit of U.S. Provisional Patent Application Ser. No. 61/002,823, filed Nov. 12, 2007, entitled DURABLE SWELLABLE HYDROGEL MATRIX AND METHODS, the disclosure of which is incorporated herein by reference.

### FIELD OF THE INVENTION

The invention is also directed to hydrogels, and compositions and methods for their preparation. The invention relates to systems and methods for the occlusion of an internal portion of the body by a hydrogel or hydrogel-associated article.

### BACKGROUND

A hydrogel is typically thought of as an insoluble matrix of crosslinked hydrophilic polymers having the capacity to absorb large amounts of water. Due to their physical properties and ability to be prepared from biocompatible materials, hydrogels have considerable use in biomedical applications. For example, hydrogels have been used as material for the treatment of wounds, as well as vehicles for the release of drugs. Hydrogels have also been used as coatings on the surface of medical devices, and can be used to improve the hydrophilicity or lubricity of the device surface.

Hydrogels are typically characterized by their capacity to swell upon absorption of water from a dehydrated state. This swelling can be affected by conditions in which the hydrogel is placed, such as by pH, temperature, and the local ion concentration and type. Several parameters can be used to define or characterize hydrogels in a swollen state, including the swelling ratio under changing conditions, the permeability coefficient of certain solutes, and the mechanical behavior of the hydrogel under conditions of its intended use.

Hydrogels that undergo a considerable degree of swelling can be useful for many medical applications in the body in which the hydrogel is placed, or is formed. However, hydrogels having a high degree of swelling may also be structurally unsuitable for use in the body. For example, considerable swelling may cause the hydrogel to become fragile, and fracture or fragment upon contact with body tissue. This could cause the hydrogel, or a device associated with the hydrogel, to lose its functionality, or could introduce complications in the body if a portion of the hydrogel is dislodged from the target site.

### SUMMARY

The present invention provides polymeric matrix-forming formulations, swellable polymeric matrices, medical articles associated with the swellable polymeric matrices, and methods of using the swellable polymeric matrices. The polymeric matrices of the invention are substantially swellable in aqueous environments to form hydrogels that are durable and well suited for use in the body. The swellable polymeric matrices are formed from a combination of polymeric materials that provide high water absorbing capacity as well as a high density of crosslinking. As such, the present invention addresses issues with swellable polymeric matrices that may demonstrate good swelling but result in hydrogels having insufficient structural properties, such as insufficient durability.

The swellable polymeric matrices of the invention are particularly useful when implanted or formed at a target site in the body. The polymeric matrices form swollen hydrogels that can occlude a target area of the body, and provide a desired biological effect at the target area. The swellable polymeric matrices can be delivered to the target area in a dry, or partially dry (dehydrated) state, where at the target area, the matrices become hydrated and swell to occlude or block the area. The occlusion or blockage can have a biological effect. For example, the occluding hydrogel can prevent the movement of biological fluids, tissue, or other biological material, across or into the occluded area.

The polymeric matrices of the present invention provide the advantage of forming swollen hydrogels with improved durability, without loss of swellability. The use of the swellable polymeric matrices as described herein can therefore provide improved function in vivo. For example, the polymeric matrices are less likely to fracture following swelling. This can provide more complete occlusion or blockage at the target area and can also increase functional lifetime of the hydrogel following implantation.

The polymeric matrices can be used alone at the target area, or can be used in association with a medical article. For example, in some aspects, the polymeric matrices can be in the form of an overmold, or in the form of a coating on an implantable medical article. The non-hydrogel portion of the article can facilitate delivery and function of the hydrogel at the target site.

The matrix can be formed from a composition that includes a combination of two, or more than two, polymeric components. The composition comprises a first polymer that is hydrophilic and that comprises a pendent reactive group, and a second polymer that is also hydrophilic, but that comprises a pendent reactive group different than the reactive group on the first polymer. The reactive groups on the first polymer and second polymer provide crosslinking through different chemical mechanisms. For example, in this aspect, crosslinking can be achieved through a combination of the activation and reaction of ethylenically unsaturated groups and photo-reactive groups.

In some cases, the first polymer can be a oxyalkylene polymer, such as poly(ethylene glycol). In some cases, the second polymer can be selected from the group consisting of poly(acrylamide), poly(methacrylamide), poly(vinylpyrrolidone), and poly(acrylic acid), or copolymers thereof.

In another aspect, the invention correspondingly provides a swellable polymeric matrix formed of a crosslinked network of polymeric material. Polymeric reagents can be used to form a biocompatible swellable or swollen polymeric matrix comprising first and second polymer-containing segments covalently linked via reacted groups, wherein the first polymer-containing segment comprises a hydrophilic polymer comprising pendent polymerized groups, and the second polymer-containing segment comprises a hydrophilic polymer comprising pendent reacted photogroups.

The combination of these two matrix-forming components provides a polymeric matrix with a particular crosslinked architecture having at least the two desirable properties of high swellability and durability. In some formations, the polymeric matrix is capable of swelling in water to a weight of about 1.5 times its weight or greater in a dehydrated form. In some formations the matrix is capable of exerting a swelling force of about 100 g/cm² or greater from a dehydrated form. In some formations, the polymeric matrix is capable of swelling in water to a size of at least about 25% greater than its size in a dehydrated form.

In another aspect, the invention provides a medical implant having a swellable polymeric matrix formed of a crosslinked network of polymeric material. The implant comprises a non-porous article having a surface on which the biocompatible swellable or swollen polymeric matrix is associated with. The matrix can be associated with the article in various ways, such as in the form of an overmold or a coating on the article.

The matrices (alone, or in association with an article) are substantially swellable and provide a durable hydrogels upon swelling. The matrix or medical implant can be configured for placement in target areas of the body, such as in aneurysms, and portions the reproductive tract, such as the fallopian tube. The matrix or medical implant can be implanted in the body when the matrix is in a dehydrated form, and during and/or following implantation, the matrix can become rehydrated and swell. In some cases, the matrix is swellable upon placement in the body to provide a diameter that is three times, or greater than three times, than the diameter of the matrix or medical implant in the dehydrated form.

In another aspect, the invention provides a method for space filling or occluding an area within the body. The method includes steps of placing at a target site in the body a biocompatible swellable matrix (alone, or in association with an article) comprising first and second polymer-containing segments covalently linked via reacted groups, wherein the first polymer-containing segment comprises a hydrophilic polymer comprising pendent polymerized groups, and second polymer-containing segment comprises a hydrophilic polymer comprising pendent reacted photogroups. The method also includes a step of allowing the matrix to swell at the target site, which occludes the target site.

### DETAILED DESCRIPTION

The embodiments of the present invention described below are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

The present invention provides improved polymeric matrices that can be swollen in situ to a durable hydrogel that blocks or occludes a target area in the body. The swellable polymeric matrices are formed from two different polymeric-based components that have pendent reactive groups. The pendent reactive groups can be activated or reacted to crosslink the components to form a swellable polymeric matrix. Other components can optionally be included for formation of the matrix.

The swellable polymeric matrix can be used in various forms. For example, the swellable polymeric matrix can be in a form of an overmold on a medical article. The matrix can also be in the form of a coating on a medical article. The swellable polymeric matrix can also be used as a medical implant itself (i.e., formed by the matrix-forming composition).

Generally, the swellable polymeric matrix is formed from a composition that includes a combination of two, or more than two, polymeric components. The composition includes a first component (a hydrophilic polymer comprising one or more pendent reactive groups), and a second component (second polymer) that is also hydrophilic, but that comprises a pendent reactive group different than the reactive group on the first component.

A “swellable polymeric matrix” refers to a crosslinked matrix of polymeric material formed from at least the first and second components. The polymeric matrix can either be dehydrated or can contain an amount of water that is less than the amount of water present in a fully swollen matrix (the fully hydrated matrix being referred to herein as a “hydrogel”). Typically, the matrix is not fully hydrated when delivered to a target site in the body for occlusion. The invention contemplates the matrix in various levels of hydration.

To facilitate discussion of the invention, polymerizable groups will be discussed as one type of the reactive groups pendent from the components that form the swellable polymeric matrix. The hydrophilic polymer (i.e., a macromer) includes one or more “polymerizable group(s)” which generally refer to chemical groups that are polymerizable in the presence of free radicals. This macromer is referred to herein at the “first component” or the “first polymer”, and forms the first polymer-containing segment in the swellable matrix. Polymerizable groups generally include a carbon-carbon double bond that can be an ethylenically unsaturated group or a vinyl group. Exemplary polymerizable groups include acrylate groups, methacrylate groups, ethacrylate groups, 2-phenyl acrylate groups, acrylamide groups, methacrylamide groups, itaconate groups, and styrene groups.

Polymers can be effectively derivatized in organic, polar, or anhydrous solvents, or solvent combinations to produce macromers. Generally, a solvent system is used that allows for polymer solubility and control over the derivatization with polymerizable groups. Polymerizable group-containing compounds, such as glycidyl acrylate, can be reacted with synthetic polymers as well as natural polymers (including polysaccharides and polypeptides) in straightforward synthetic processes. In some aspects, the polymerizable group is present on the macromer at a molar ratio of 0.002 μmol or greater of polymerizable group (such as an acrylate group) per 1 mg of macromer. In some aspects the macromer is derivatized with polymerizable groups in amount in the range from about 0.05 μmol to about 2 μmol of polymerizable group (such as an acrylate group) per 1 mg of macromer.

Polymers and macromers used for making the swellable polymeric matrices of the invention can be described in terms of molecular weight. “Molecular weight,” as used herein, more specifically refers to the “weight average molecular weight” or M_{w}, which is an absolute method of measuring molecular weight and is particularly useful for measuring the molecular weight of a polymer (preparation), such as macromer preparations. Polymer preparations typically include polymers that individually have minor variations in molecular weight. In some cases, the polymers have a relatively higher molecular weight (e.g., versus smaller organic compounds) and such minor variations within the polymer preparation do not affect the overall properties of the polymer preparation. The weight average molecular weight (M_{w}) can be defined by the following formula:

$${M}_{w} = \frac{{\sum }_{:} {N}_{i} {M}_{i}^{2}}{{\sum }_{:} {N}_{i} {M}_{i}}$$

wherein N represents the number of moles of a polymer in the sample with a mass of M, and Σᵢ is the sum of all NᵢMᵢ (species) in a preparation. The M_{w} can be measured using common techniques, such as light scattering or ultracentrifugation. Discussion of M_{w} and other terms used to define the molecular weight of polymer preparations can be found in, for example, Allcock, H. R. and Lampe, F. W., Contemporary Polymer Chemistry; pg 271 (1990).

The first component (a macromer) can be formed from a biocompatible polymer that is hydrophilic. In some cases the macromer is based on a linear hydrophilic polymer. Exemplary polymers that that can be used to form the first component can be based on one or more of the following polymers: poly(vinylpyrrolidone) (PVP), poly(ethylene oxide) (PEO), poly(ethyloxazoline), poly(propylene oxide) (PPO), poly(meth)acrylamide (PAA) and poly(meth)acylic acid, poly(ethylene glycol) (PEG) (see, for example, U.S. Pat. Nos. 5,410,016, 5,626,863, 5,252,714, 5,739,208 and 5,672,662) PEG-PPO (copolymers of polyethylene glycol and polypropylene oxide), hydrophilic segmented urethanes (see, for example, U.S. Pat. Nos. 5,100,992 and 6,784,273), and polyvinyl alcohol (see, for example, U.S. Pat. Nos. 6,676,971 and 6,710,126).

In some aspects, the first component has a molecular weight in the range of 250 Da to 40 kDa.

In some aspects, the macromer is formed from an oxyalkylene polymer, such as an ethylene glycol polymer or oligomer having the structure HO—(CH₂—CH₂—O)ₙ—H. As an example, the value of n ranges from about 3 to about 150 and the number average molecular weight (Mn) of the poly(ethylene glycol) ranges from about 250 Da to about 40 kDa, more typically ranging from about 300 Da to about 20 kDa, from about 400 Da to about 10 kDa, from about 500 Da to about 5000 Da, or about 600 Da to about 1000 Da.

An oxyalkylene polymer can be effectively derivatized to add polymerizable groups to produce oxyalkylene based macromers. Polymerizable groups such as glycidyl acrylate, glycidyl methacrylate, acrylic or methacrylic acid can be reacted with the terminal hydroxyl groups of these polymers to provide terminal polymerizable groups.

Some specific examples of alkylene oxide polymer-based macromers include, poly(propylene glycol)₅₄₀-diacrylate, poly(propylene glycol)₄₇₅-dimethacrylate, poly(propylene glycol)₉₀₀-diacrylate, poly(ethylene glycol)₂₅₀-diacrylate, poly(ethylene glycol)₅₇₅-diacrylate, poly(ethylene glycol)₅₅₀-dimethacrylate, poly(ethylene glycol)₇₅₀-dimethacrylate, poly(ethylene glycol)₇₀₀-diacrylate, and poly(ethylene glycol)₁₀₀₀-diacrylate, poly(ethylene glycol)₂₀₀₀ diacrylate, poly(ethylene glycol)₁₀₀₀ monomethyl ether monomethacrylate, and poly(ethylene glycol)₅₀₀ monomethyl ether monomethacrylate. These types of alkylene oxide polymer-based macromers are available from Sigma-Aldrich (St. Louis, Mo.) or Polysciences (Warrington, Pa.).

In some aspects, the first component comprises a non-linear or branched compound comprising two or more hydrophilic polymeric portions and pendent polymerizable groups. For example, the non-linear or branched compound can include polymerizable groups pendent from the polymeric portions of the compound. In these cases, the compound can also be considered a macromeric compound.

A “non-linear” or “branched” compound having polymeric portions refers to those having a structure different than a linear polymer (which is a polymer in which the molecules form long chains without branches or cross-linked structures). Such a compound can have multiple polymeric “arms” which are attached to a common linking portion of the compound. Non-linear or branched compounds are exemplified by, but not limited to, those having the following general structures:

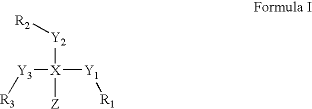

wherein X is a linking atom, such as one selected from C or S, or a linking structure, such a homo- or heterocyclic ring; to Y₁ to Y₃ are bridging groups, which can independently be, for example, —Cₙ—O—, wherein n is 0 or an integer of 1 or greater; R₁ to R₃ are independently hydrophilic polymeric portions, which can be the same or different, and have one or more pendent polymerizable groups; and Z is a non-polymeric group, such as a short chain alkyl group.

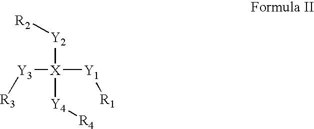

wherein X is a linking atom, such as one selected from C or S, or a linking structure, such a homo- or heterocyclic ring; to Y₁ to Y₄ are bridging groups, which can individually be, for example, —Cₙ—O—, wherein n is 0 or an integer of 1 or greater; and R₁ to R₄ independently hydrophilic polymeric portions, which can be the same or different, and have one or more pendent polymerizable groups.

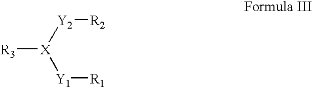

wherein X is a linking atom or group, such as one selected from N, C—H, or S—H, or a linking structure, such a homo- or heterocyclic ring; to Y₁ and Y₂ are bridging groups, which can individually be, for example, —Cₙ—O—wherein n is 0 or an integer of 1 or greater; and R₁ to R₃ independently hydrophilic polymeric portions, which can be the same or different, and have one or more pendent polymerizable groups.

The non-linear or branched compound can be prepared from a polyol, such as a low molecular weight polyol (for example, a polyol having a molecular weight of 200 Da or less). In some aspects the non-linear or branched compound can be derived from a triol, a tetraol, or other multifunctional alcohol. Exemplary polyol derivatives include derivatives of pentaerythritol, trimethylolpropane, and glycerol.

The polymeric portions of the non-linear or branched compound can be selected from PVP, PEO, poly(ethyloxazoline), PPO, PAA and poly(meth)acylic acid, PEG, and PEG-PPO, hydrophilic segmented urethanes, and polyvinyl alcohol, such as those described herein.

In some aspects, the non-linear or branched compound comprises one or more polymeric portions that is or are an oxyalkylene polymer, such as an ethylene glycol polymer.

For example, the preparation of a PEG-triacrylate macromer (trimethylolpropane ethoxylate (20/3 EO/OH) triacrylate macromer), which can be used as a non-linear or branched compound, is described in Example 5 of commonly assigned U.S. Patent Application Publication No. 2004/0202774A1 (Chudzik, et al.).

In some aspects, the non-linear or branched compound has a molecular weight in the range of about 300 Da to about 20 kDa, or more specifically in the range of about 500 Da to about 2500 Da.

The second polymer that is hydrophilic comprises a pendent reactive group (such as a photo-reactive group) that is different than the reactive group on the first component. This polymeric component is the second component, and forms the second polymer-containing segment in the swellable matrix. In aspects, the second polymer comprises non-oxyalkylene monomeric units, such as monomeric units selected from (meth)acrylamide, vinyl pyrrolidone, and (meth)acrylic acid. The second polymer can be a homopolymer, such as polyacrylamide, polymethacrylamide, polyvinylpyrrolidone, polyacrylic acid, or polymethacrylic acid having pendent photo-reactive groups.

The second polymer can also be a copolymer. For example, the copolymer can comprise non-oxyalkylene monomeric units, such as monomeric units selected from (meth)acrylamide, vinyl pyrrolidone, and (meth)acrylic acid. In some aspects the amount of non-oxyalkylene monomeric units, such as one or more monomeric units selected from (meth)acrylamide, vinyl pyrrolidone, and (meth)acrylic acid, are in the copolymer in an amount of about 50% by weight or greater, about 75% by weight or greater, or about 80% by weight or greater.

Some exemplary copolymers comprise non-oxyalkylene and oxyalkylene monomeric units. Exemplary oxyalkylene monomeric units include alkylene oxides selected from ethylene glycol and propylene glycol. In some aspects, the amount of oxyalkylene monomeric units are in the copolymer in an amount of about 50% by weight or less, about 25% by weight or less, or about 20% by weight or less, such as in the range of about 1% by weight to about 20% by weight, or about 1% by weight to about 15% by weight.

The copolymer can also include amphiphilic monomeric units. Exemplary amphiphilic monomers are selected from the group consisting of diacetone acrylamide (DAA), vinyloxyethanol (VOE), 2-acrylamido-2-methylpropane (AMPS), and methyl acryloyl lactate (ALM). In some aspects the amount of amphiphilic monomeric units are in the copolymer in an amount of about 20% by weight or less, about 10% by weight or less, or about 0.1% by weight to about 10% by weight.

The copolymer can also be structured as a block copolymer and can include, for example, polyacrylamide, polymethacrylamide, polyvinylpyrrolidone, polyacrylic acid, polyethylene glycol, polyvinyl alcohol, and poly(HEMA) blocks.

The second polymer can be substantially larger than the first polymer. This can form a matrix wherein the molecular weight of the first polymer-containing segment is less than the second polymer-containing segment. For example, the matrix can be formed using a first polymer (macromer) that has a molecular weight of less then 20 kDa (such as in the range of about 300 Da to about 20 kDa, or about 300 Da to about 5 kDa), and a second polymer (photo-polymer) that has a molecular weight of greater than 20 kDa (such as in the range of about 25 kDa to about 10⁷ kDa, or about 25 kDa to about 10⁶ kDa).

As mentioned, the first polymer can include a polymerizable group, and the second polymer accordingly includes a reactive group that is different than the polymerizable group. One exemplary class of reactive groups that different than polymerizable groups, and which can be pendent from the second polymer include photoreactive groups.

A “photoreactive group” includes one or more reactive moieties that respond to a specific applied external energy source, such as radiation, to undergo active species generation. For example, the photoreactive group can be activated to an active specie such as a nitrene, carbene, or an excited ketone state, with resultant covalent bonding to an adjacent targeted chemical structure. Examples of such photoreactive groups are described in U.S. Pat. No. 5,002,582 (Guire et al., commonly owned by the assignee of the present invention), the disclosure of which is incorporated herein in its entirety. Photoreactive groups can be chosen to be responsive to various portions of the electromagnetic spectrum, typically ultraviolet, visible or infrared portions of the spectrum. “Irradiation” refers to the application of electromagnetic radiation to a surface.

Photoreactive aryl ketones are preferred photoreactive groups on the photoreactive polymer, and can be, for example, acetophenone, benzophenone, anthraquinone, anthrone, and anthrone-like heterocycles (i.e., heterocyclic analogs of anthrone such as those having N, O, or S in the 10-position), or their substituted (e.g., ring substituted) derivatives. Examples of aryl ketones include heterocyclic derivatives of anthrone, including acridone, xanthone and thioxanthone, and their ring substituted derivatives. As an example, thioxanthone, and its derivatives, having excitation wavelengths greater than about 360 nm.

The azides are also a suitable class of photoreactive groups on the photoreactive polymer and include arylazides (C₆R₅N₃) such as phenyl azide and particularly 4-fluoro-3-nitrophenyl azide, acyl azides (—CO—N₃) such as ethyl azidoformate, phenyl azidoformate, sulfonyl azides (—SO₂—N₃) such as benzensulfonyl azide, and phosphoryl azides (RO)₂PON₃ such as diphenyl phosphoryl azide and diethyl phosphoryl azide.

Diazo compounds constitute another suitable class of photoreactive groups on the photoreactive polymers and include diazoalkanes (—CHN₂) such as diazomethane and diphenyldiazomethane, diazoketones (—CO—CHN₂) such as diazoacetophenone and 1-trifluoromethyl-1-diazo-2-pentanone, diazoacetates (—O—CO—CHN₂) such as t-butyl diazoacetate and phenyl diazoacetate, and beta-keto-alpha-diazoacetates (—CO—CN₂—CO—O—) such as 3-trifluoromethyl-3-phenyldiazirine, and ketenes (—CH═C═O) such as ketene and diphenylketene.

Exemplary photoreactive groups are shown as follows.

TABLE 1

| Photoreactive Group | Bond Formed |
|---|---|
| aryl azides | Amine |
| acyl azides | Amide |
| Azidoformates | Carbamate |
| sulfonyl azides | Sulfonamide |
| phosphoryl azides | Phosphoramide |
| Diazoalkanes | new C—C bond |
| Diazoketones | new C—C bond and ketone |
| Diazoacetates | new C—C bond and ester |
| beta-keto-alpha- | new C—C bond and beta-ketoester |
| diazoacetates | |
| aliphatic azo | new C—C bond |
| Diazirines | new C—C bond |
| Ketenes | new C—C bond |
| photoactivated ketones | new C—C bond and alcohol |

The photoreactive groups of the photoreactive polymer can allow the formation of a covalent bond between the first polymer and the photoreactive polymer. Therefore, the reacted photoreactive groups serve to crosslink polymeric strands together, allowing the formation of a network of covalently crosslinked polymeric strands.

Optionally, other materials, such as other macromers, can be used to form the swellable biodegradable polymeric matrix. These may be referred to as “third macromer,” “fourth macromer,” etc. If any additional macromers are used to form the matrix, these may be biodegradable, or biostable.

In one aspect, the matrix is formed from a composition that includes the first polymer (macromer), wherein the first polymer is linear and hydrophilic, a second polymer (photo-polymer), and a third component that comprises a non-linear or branched component. The third non-linear or branched component can be one that is described herein, such as a branched component that has multiple oxyalkylene polymeric arms. The third non-linear or branched component can increase the crosslinking density in the matrix and provide a more durable hydrogel. In some aspects, the third non-linear or branched component can be used in an amount that is less that that of the first and/or second polymers. For example, the third component can be used in an amount of about 50% or less of the amount (weight) of the first polymer, or about 25% or less, such as in the range of about 1% to about 20% of the amount of the first polymer.

Alternatively, the matrix can be formed using a biodegradable polymer. The biodegradable polymer can be used in amount that allows for the degradation of the implanted matrix after a period of time in the body. For example, the matrix can be prepared to provide an occlusion function for a predetermined period of time at the target site, and then degrade after the occlusive function is no longer needed.

For example, the matrix can be prepared to include a biodegradable macromer. The macromer can be enzymatically and/or hydrolytically degradable, thereby providing the matrix with polymeric segments that can be broken down in the body by an enzymatic and/or non-enzymatic mechanism(s). Degradation of a polymeric segment that includes a degradable portion can result in loss of the matrix structure by surface or bulk erosion.

One exemplary biodegradable macromer that can be used to form the swellable polymeric matrices is based on poly-α(1→4)glucopyranose. A α(1→4)glucopyranose polymer includes repeating glucopyranose monomeric units having α(1→4) linkages that are capable of being enzymatically degraded. Exemplary α(1→4)glucopyranose polymers include maltodextrin, amylose, cyclodextrin, and polyalditol. Modification of a α(1→4)glucopyranose polymer, such as amylose or maltodextrin, to provide pendent polymerizable groups can be carried out using known techniques. In some modes of preparation, a portion of the hydroxyl groups (which are naturally pendent from α(1→4)glucopyranose polymer) are reacted with a compound having a hydroxyl-reactive group and a polymerizable group. For example, commonly assigned patent application, published as U.S. Pub No. 2007/0065481 (Chudzik et al.) describes modification of α(1→4)glucopyranose polymers to provide pendent acrylate and methacrylate groups.

The matrix formed from the first polymer and second polymer can have an architecture wherein first polymers are crosslinked via their polymerized groups, with the second polymer at least crosslinked to the crosslinked first polymers or other second polymers via reacted photo groups.

The swellable matrix can be prepared with a desired ratio of first polymer-containing segment to second polymer-containing segment. In some aspects, the first polymer-containing segment and the second polymer-containing segment are present in the matrix at a weight ratio in the range of 9:1 to 1:3, respectively. In more specific aspects the first polymer-containing segment and the second polymer-containing segment are present in the matrix at a weight ratio in the range of 4:1 to 1:2, respectively.

A composition can be prepared containing the first compound (the macromer based on a hydrophilic polymer) and the second compound (the second polymer being hydrophilic and that comprises reactive groups that are different than the reactive groups of the first polymer) at concentrations sufficient to form the polymeric matrix that can be swollen to a durable hydrogel.

The composition including the first and second components can have a viscosity that is suitable for the type of matrix-forming process performed. In order to prepare a composition, the first and second components (and any other component), can be dissolved or suspended in a suitable polar liquid. Exemplary polar liquids include alcohol or water. Combinations of polar solvents can also be used. In some aspects, the viscosity of the composition is in the range of about 5 to 200 cP (at about 25° C.).

In many modes of practice the first polymer (macromer) is present in the composition used to form the swellable matrix at a concentration in the range of about 50 mg/mL to about 300 mg/mL, about 50 mg/mL to about 250 mg/mL, about 50 mg/mL to about 200 mg/mL, or about 50 mg/mL to about 150 mg/mL. In some modes of practice, the first polymer (macromer) is used in an amount of about 100 mg/mL.

The second polymer (photo-polymer) can be used in combination with the first polymer (macromer) at a concentration sufficient to form a swellable polymeric matrix. In many modes of practice the second polymer (photo-polymer) is present in the composition used at a concentration in the range of about 5 mg/mL to about 250 mg/mL, about 10 mg/mL to about 150 mg/mL, about 15 mg/mL to about 100 mg/mL, or about 20 mg/mL to about 80 mg/mL. In some modes of practice, the second polymer (photo-polymer) is used in an amount of about 60 mg/mL.

Another way of describing the matrix-forming composition is by reference to the total amount matrix-forming materials in the composition. In many modes of practice the total amount of matrix forming material (including the first and second polymers, and optionally any third polymer (macromer), etc.) is in the range of about 75 mg/mL to about 400 mg/mL, about 100 mg/mL to about 300 mg/mL, about 100 mg/mL to about 250 mg/mL, or about 100 mg/mL to about 200 mg/mL. In some modes of practice, the total amount of matrix forming material in the composition is about 130 mg/mL.

In some aspects, the composition includes an initiator that is capable of promoting the formation of a reactive species from a polymerizable group. For example, the initiator can promote a free radical reaction of hydrophilic polymer having pendent polymerizable groups. In one embodiment the initiator is a compound that includes a photoreactive group (photoinitiator). For example, the photoreactive group can include an aryl ketone photogroup selected from acetophenone, benzophenone, anthraquinone, anthrone, anthrone-like heterocycles, and derivatives thereof.

In some aspects the photoinitiator includes one or more charged groups. The presence of charged groups can increase the solubility of the photoinitiator (which can contain photoreactive groups such as aryl ketones) in an aqueous system. Suitable charged groups include, for example, salts of organic acids, such as sulfonate, phosphonate, carboxylate, and the like, and onium groups, such as quaternary ammonium, sulfonium, phosphonium, protonated amine, and the like. According to this embodiment, a suitable photoinitiator can include, for example, one or more aryl ketone photogroups selected from acetophenone, benzophenone, anthraquinone, anthrone, anthrone-like heterocycles, and derivatives thereof; and one or more charged groups. Examples of these types of water-soluble photoinitiators have been described in U.S. Pat. No. 6,278,018.

Water-soluble polymerization initiators can be used at a concentration sufficient to initiate polymerization of the first and second components and formation of the matrix. For example, a water-soluble photo-initiator as described herein can be used at a concentration of about 0.5 mg/mL or greater. In some modes of practice, the photo-initiator is used at a concentration about 1.0 mg/mL along with the matrix-forming components.

Generally, matrix formation is initiated by subjecting the matrix-forming composition (which includes photoreactive groups) to actinic radiation in an amount that promotes activation of the photoreactive groups. Activation of the photoreactive groups can initiate polymerization of the macromer component, and can also promote covalent bonding of the photogroups on the photopolymer to a target (such as another polymer in the composition).

Actinic radiation can be provided by any suitable light source that promotes activation of the photoreactive groups. Preferred light sources (such as those available from Dymax Corp.) provide UV irradiation in the range of 190 nm to 360 nm. Filters can be used in connection with the step of activating the photoreactive groups. The use of filters can be beneficial from the standpoint that they can selectively minimize the amount of radiation of a particular wavelength or wavelengths. This can be beneficial if one or more components of the coating are sensitive to radiation of a particular wavelength(s), and that may degrade or decompose upon exposure.

In some aspects, a “pre-formed” swellable polymeric matrix is prepared, referring to those matrices that are not formed in situ, but rather away from a tissue site. A pre-formed swellable polymeric matrix can have a defined structure. A pre-formed matrix can be created using a mold or casting so the matrix can be made into a particular shape. Alternatively, a pre-formed matrix can be created and then shaped as desired, by a process such as cutting. Exemplary shapes useful for tissue treatment include, but are not limited to, spherical, cylindrical, clam-shell, flattened, rectangular, square, and rounded shapes.

In some aspects of the invention, the swellable polymeric matrix is formed in association with a medical article. For example, the matrix can be formed as an overmold or a coating in association with a part of, or the entire article.

A “coating” refers to one or more layers of matrix material, formed by applying the matrix forming materials to all or a portion of a surface of an article by conventional coating techniques.

An “overmold” refers to matrix material formed in association with all or a portion of a surface of an article. An overmold of matrix material is generally thicker than a coating, and typically formed using a molding process rather than a coating process.

A “medical article” or “medical device” refers to an article used in a medical procedure. Typically, the matrix is associated with a surface of the article, such as formed on a surface of an implantable medical device. From a structural standpoint, the implantable medical article may be a simple article, such as a rod, pellet, sphere, or wire, on which the swellable matrix can be formed. The implantable medical article can also have a more complex structure or geometry, as would be found in an intralumenal prosthesis, such as a stent.

In aspects, the article is non-porous. A non-porous article has a solid structure that does not allow the infiltration or ingrowth of cells into the structure of the article.

An medical implant having a swellable polymeric matrix (formed using the hydrogel-forming materials of the invention), or a portion thereof, can be configured to be placed within the vasculature (an implantable vascular device), such as in an artery, vein, fistula, or aneurysm. In some cases the medical implant includes an occlusion device selected from vascular occlusion coils, wires, or strings that can be inserted into aneurysms. Some specific vascular occlusion devices include detachable embolization coils. In some cases the medical implant comprises a stent.

Implantable medical articles can be prepared from metals such as platinum, gold, or tungsten, although other metals such as rhenium, palladium, rhodium, ruthenium, titanium, nickel, and alloys of these metals, such as stainless steel, titanium/nickel, and nitinol alloys, can be used.

The surface of metal-containing medical articles can be pretreated (for example, with a Parylene™-containing coating composition) in order to alter the surface properties of the biomaterial, when desired. Metal surfaces can also be treated with silane reagents, such as hydroxy- or chloro-silanes.

Implantable medical articles can also be partially or entirely fabricated from a plastic polymer. In this regard, the swellable polymeric matrix can be formed on a plastic surface. Plastic polymers include those formed of synthetic polymers, including oligomers, homopolymers, and copolymers resulting from either addition or condensation polymerizations. Examples of suitable addition polymers include, but are not limited to, acrylics such as those polymerized from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; vinyls such as ethylene, propylene, vinyl chloride, vinyl acetate, vinyl pyrrolidone, vinylidene difluoride, and styrene. Examples of condensation polymers include, but are not limited to, nylons such as polycaprolactam, polylauryl lactam, polyhexamethylene adipamide, and polyhexamethylene dodecanediamide, and also polyurethanes, polycarbonates, polyamides, polysulfones, poly(ethylene terephthalate), polydimethylsiloxanes, and polyetherketone.

A medical implant with a swellable polymeric matrix “overmold” can be formed in a process using a mold, a composition comprising the matrix-forming material, and a medical article. The medical article can be placed in a portion of the mold so the composition can be placed in contact with all or a portion of the surface of the article. For example, a article in the shape of a rod or coil is fixtured in a mold so that that composition can be in contact with the entire surface of the article. The composition can then be added to mold and treated to promote matrix formation. In some cases, the mold is made of a material that allows UV light to pass through it, and the composition can include a photo-initiator, which is activated by the UV and causes matrix formation.

In another exemplary mode of preparation, a matrix overmold can be formed by adding the composition to the mold and then partially polymerizing the matrix so the composition increases in viscosity. The medical article can then be placed in the partially polymerized composition, and due to its increased viscosity, suspends the article within the composition as desired. The composition can then be fully polymerized to solidify the materials of the composition, which forms the swellable polymeric matrix as an overmold on the article. After the swellable polymeric matrix forms as an overmold, the formed medical implant can be removed from the mold.

The weight of the polymeric matrix can be a substantial percentage of the weight of the overall medical implant. When the matrix is in a partially hydrated for fully hydrated state, it can have a weight that is substantially greater than the article which it overmolds.

The overmold can be formed on any desired medical article and can dimensions suitable for occluding a target site in the body. The swellable polymeric matrix in an overmold is typically thicker than the matrix of a coating, which can provide advantages for occlusion of a target site.

In some aspects, the matrix has a thickness in the range of about 50 μm to about 500 μm, and more specifically in the range of about 100 μm to about 300 μm. The matrix can then be dried to have a thickness in the range of about 25 μm to about 400 μm, respectively, and more specifically in the range of about 75 μm to about 250 μm, respectively. The matrix can be hydrated (e.g., in vivo), which can swell the matrix to a thickness in the range of about 100 μm to about 2500 μm, respectively, and more specifically in the range of about 750 μm to about 1500 μm, respectively.

As a specific example, in the case of a fallopian tube occlusion coil having a diameter of about 0.5 mm, a swellable polymeric matrix in the form of an overmold is formed on the coil. The overmold has a thickness in the range of about 100 μm to about 450 μm in a dried state. During and/or after delivery of the article to the fallopian tube, the coating swells to have a thickness in the range of about 750 μm to about 1500 μm, causing occlusion of the fallopian tube and prevention of fertilization.

A medical implant with an overmolding can be delivered to a target site in the body, where it hydrates to a hydrogel within the target site. Delivery of the device can be performed using a catheter and/or other guide instruments, such as guidewires.

The swellable polymeric matrix can become hydrated in a relatively short period of time, such as period of time in the range of about 30 minutes to about 2 hours, or about 1 hour. Swelling of the polymeric matrix can be monitored to determine if the hydrogel occludes the target site as desired.

In another aspect, the swellable polymeric matrix is in the form of a coating on a medical article. A matrix coating that includes the first and second compounds can be formed various ways.

In one mode of practice, a composition including the first and second compounds is dip-coated onto the surface of the substrate to form a coating. The composition on the surface can then be treated to cause matrix formation. For example, a composition including the first and second compounds, and a photoactivate polymerization initiator is dipcoated on the surface of a device. During and/or after the dip-coating step, the applied material can be irradiated to promote polymerization of the first and second components, and matrix formation.

Other techniques, such as brushing or spraying the composition can be used to form the coating. The method of spray coating can be performed by spraying the composition on the surface the article, and then treating the composition to form the coating.

In another aspect of the invention, the (first) linear hydrophilic polymer, and a (second) non-linear or branched compound comprising two or more hydrophilic polymeric portions, each having pendent reactive groups, is used to form a polymeric matrix article which is capable of swelling to a hydrogel. A device such as one used in an overmolding process is not used as a portion of the article, and the swellable matrix itself forms the medical implant.

Such an implantable matrix article can have a simple or a complex geometry. A simple geometry is exemplified by an implant that is in the form of a filament (e.g., threads, strings, rods, etc.). A matrix implant with a simple geometry can be prepared by various methods. One method for forming the matrix implant uses the same process as used to form the overmolded device, but does not include an article within the mold. Again, the mold can be, for example, a piece of tubing which has an inner area corresponding to the first configuration of the body member. The composition can then be injected into the tubing to fill the tubing. The composition in the tubing can then be treated to activate the polymerization initiator (such as by photo-initiated polymerization). Polymerization promotes crosslinking of the first and second components (and any other optional polymerizable material) and establishes a polymeric matrix in the configuration of the mold.

In many cases, the matrix article can be used in the same way that the overmolded article is used.

The swellable polymeric matrices of the present invention can also include a bioactive agent, releasable from, and/or stable in the hydrogel. Examples of bioactive agents that can be included in the hydrogel include: ACE inhibitors, actin inhibitors, analgesics, anesthetics, anti-hypertensives, anti polymerases, antisecretory agents, anti-AIDS substances, antibiotics, anti-cancer substances, anti-cholinergics, anti-coagulants, anti-convulsants, anti-depressants, anti-emetics, antifungals, anti-glaucoma solutes, antihistamines, antihypertensive agents, anti-inflammatory agents (such as NSAIDs), anti metabolites, antimitotics, antioxidizing agents, anti-parasite and/or anti-Parkinson substances, antiproliferatives (including antiangiogenesis agents), anti-protozoal solutes, anti-psychotic substances, anti-pyretics, antiseptics, anti-spasmodics, antiviral agents, calcium channel blockers, cell response modifiers, chelators, chemotherapeutic agents, dopamine agonists, extracellular matrix components, fibrinolytic agents, free radical scavengers, growth hormone antagonists, hypnotics, immunosuppressive agents, immunotoxins, inhibitors of surface glycoprotein receptors, microtubule inhibitors, miotics, muscle contractants, muscle relaxants, neurotoxins, neurotransmitters, polynucleotides and derivatives thereof, opioids, photodynamic therapy agents, prostaglandins, remodeling inhibitors, statins, steroids, thrombolytic agents, tranquilizers, vasodilators, and vasospasm inhibitors. One or more bioactive agents can be present in the polymeric matrix in an amount sufficient to provide a biological response.

In some aspects of the invention, the matrix includes a bioactive agent that is a macromolecule. Exemplary macromolecules can be selected from the group consisting of polynucleotides, polysaccharides, and polypeptides. In some aspects the bioactive agent has a molecular weight of about 1000 Da or greater.

One class of bioactive agents that can be released from the matrix includes polynucleotides. As used herein “polynucleotides” includes polymers of two or more monomeric nucleotides. Nucleotides can be selected from naturally occurring nucleotides as found in DNA (adenine, thymine, guanine, and cytosine-based deoxyribonucleotides) and RNA (adenine, uracil, guanine, and cytosine-based ribonucleotides), as well as non-natural or synthetic nucleotides.

Types of polynucleotides that can be released from the matrix include plasmids, phages, cosmids, episomes, integratable DNA fragments, antisense oligonucleotides, antisense DNA and RNA, aptamers, modified DNA and RNA, iRNA (immune ribonucleic acid), ribozymes, siRNA (small interfering RNA), miRNA (micro RNA), locked nucleic acids and shRNA (short hairpin RNA).

If it is desired to include a bioactive agent in the matrix, one of various methods can be performed to provide form a bioactive agent-containing matrix. For example, in some modes of practice, the bioactive agent is dissolved in a matrix-forming composition and then the macromers of the composition are polymerized, entrapping the bioactive agent in the matrix. Following placement at a target site in the body, the bioactive agent can be released by diffusion of the bioactive agent out of the matrix, or by degradation if the matrix is prepared from a degradable polymer.

In another mode of practice, the bioactive agent is suspended in the matrix forming composition, and the macromers of the composition are polymerized, which also results of the bioactive agent being entrapped in the matrix.

In some cases, the bioactive agent can be present in the matrix in particulate form. “Particulate form,” generally refers to small particles of bioactive agent. Small particles of bioactive agent can be formed by processes such as micronizing, milling, grinding, crushing, and chopping a solid mass of bioactive agent. Particulates of bioactive agent can be from a powdered composition of the bioactive agent. In some cases, powders of bioactive agent can be formed from processes including precipitation and/or crystallization, and spray drying. Particulates of bioactive agent can be in the form of microparticles or microspheres. The microparticles of bioactive agent can comprise any three-dimensional structure that can be immobilized in the matrix formed by the macromers described herein. Microspheres are microparticles that are spherical or substantially spherical in shape.

Bioactive agent-containing microparticles can be formed substantially or entirely of bioactive agent, or the microparticles can include a combination of a bioactive agent and a non-active agent, such as an excipient compound or polymeric material.

Microparticles formed solely of one or more bioactive agents have been described in the art. For example, the preparation of paclitaxel microparticles has been described in U.S. Pat. No. 6,610,317. Therefore the bioactive agent can be a low molecular weight compound present. As another example, the microparticle is formed from a macromolecular compound, such as a polypeptide. Polypeptide microparticles are described in commonly-assigned copending U.S. patent application Ser. No. 12/215,504 filed Jun. 27, 2008, (Slager, et al.).

In some aspects, the swellable polymeric matrix can also include a pro-fibrotic agent. A pro-fibrotic agent can promote a rapid and localized fibrotic response in the vicinity of the hydrogel. This can lead to the accumulation of clotting factors and formation of a fibrin clot in association with the hydrogel. In some aspects the pro-fibrotic agent is a polymer. The polymer can be based on a natural polymer, such as collagen, or a synthetic polymer. For example, a collagen can include polymerizable groups and can be reacted along with the first polymer (macromer) and the second polymer to be covalently incorporated into the matrix. An example of a matrix protein-based macromer is a collagen macromer, which is described in Example 12 of commonly assigned U.S. Pub. No. US-2006/0105012A1.

Alternatively, a pro-fibrotic agent can be covalently coupled to a polymeric segment via a reacted photogroup. For example, photogroup-derivatized matrix proteins, such as photo-collagen (described in commonly-assigned U.S. Pat. No. 5,744,515) and activated to bond collagen to the polymeric material forming the matrix.

The swellable polymeric matrix can also include an imaging material. Imaging materials can facilitate visualization of the polymeric matrix one implanted or formed in the body. Medical imaging materials are well known. Exemplary imaging materials include paramagnetic material, such as nanoparticular iron oxide, Gd, or Mn, a radioisotope, and non-toxic radio-opaque markers (for example, cage barium sulfate and bismuth trioxide). Radiopacifiers (such as radio opaque materials) can be included in a composition used to make the matrix. The degree of radiopacity contrast can be altered by controlling the concentration of the radiopacifier within the matrix. Common radio opaque materials include barium sulfate, bismuth subcarbonate, and zirconium dioxide. Other radio opaque materials include cadmium, tungsten, gold, tantalum, bismuth, platinum, iridium, and rhodium.

Paramagnetic resonance imaging, ultrasonic imaging, x-ray means, fluoroscopy, or other suitable detection techniques can detect the swellable or swollen matrices that include these materials. As another example, microparticles that contain a vapor phase chemical can be included in the matrix and used for ultrasonic imaging. Useful vapor phase chemicals include perfluorohydrocarbons, such as perfluoropentane and perfluorohexane, which are described in U.S. Pat. No. 5,558,854; other vapor phase chemicals useful for ultrasonic imaging can be found in U.S. Pat. No. 6,261,537.

Testing can be carried out to determine mechanical properties of the hydrogel. Dynamic mechanical thermal testing can provide information on the viscoelastic and rheological properties of the hydrogel by measuring its mechanical response as it is deformed under stress. Measurements can include determinations of compressive modulus, and shear modulus. Key viscoeslatic parameters (including compressive modulus and sheer modulus) can be measured in oscillation as a function of stress, strain, frequency, temperature, or time. Commercially available rheometers (for example, available from (TA Instruments, New Castle, Del.) can be used to make these measurements. The testing of hydrogels for mechanical properties is also described in Anseth et al. (1996) Mechanical properties of hydrogels and their experimental determination, Biomaterials, 17:1647.

The hydrogel can be measured to determine its complex dynamic modulus (G*): G*=G′+iG″=σ*/γ*, where G′ is the real (elastic or storage) modulus, and G″ is the imaginary (viscous or loss) modulus, these definitions are applicable to testing in the shear mode, where G refers to the shear modulus, σ to the shear-stress, and γ to the shear strain.

The hydrogels of the present invention can have a compressive modulus, such as greater than 500 kPa, or greater than 2000 kPa.

The hydrogel can also be measured for its swelling (or osmotic) pressure. Commercially available texture analyzers (for example, available from Stable Micro Systems; distributed by Texture Technologies Corp; Scarsdale, N.Y.) can be used to make these measurements. Texture analyzers can allow measurement of force and distance in tension or compression.

In some modes of practice, the hydrogels having swelling pressures of about 10 kPa (about 100 g/cm²) or greater, such as in the range of about 10 kPa to about 750 kPa (about 7600 g/cm²), or about 10 kPa to 196 kPa (2000 g/cm²) are used. In other words, the matrix is capable of exerting a swelling force in these ranges upon hydration from a dehydrated or partially hydrated form.

In some formations, the polymeric matrix is capable of swelling in water to a weight in the range of about 1.5 or greater its weight in a dehydrated form, such as in the range of about 1.5 to about 10 times its weight in a dehydrated form.

In some formations, the polymeric matrix is capable of swelling in water to a size that is at least 25% greater than its size in a dehydrated form, such as in the range of about 25% greater to about 150% greater than its size in a dehydrated form, or about 45% greater to about 80% greater than its size in a dehydrated form.

### EXAMPLE 1

### Poly(Ethylene Glycol) Diacrylate and Photo-PA-PEG-AMPS Crosslinkable Polymers for Gel Formation

A polymerized gel was prepared from a combination of a PEG macromer and a photopolymer.

The following process was carried out to provide a polymer with the following properties: Acetylated PA-(10% w/w)methoxy-PEG1000-MMA-(5%)AMPS-(1%)APMA-(0.015)BBA, which was subsequently used a component for matrix formation.

The photoderivatized polymer photo-PA-PEG-AMPS was prepared by a copolymerization of acrylamide, methoxy poly(ethylene glycol), 2-acrylamide-2-methylpropanesulfonic acid (AMPS), and N-(3-aminopropyl)methacrylamide (APMA) using water as the solvent. Purification of the polymer after polymerization was performed using dialysis. Photo-loading was performed secondly using BBA (4-benzoylbenzoyl chloride) in a mixed aqueous/organic solvent under Schotten-Baumann conditions. Residual amines left after the photoderivatization were capped using acetic anhydride. Final purification was done using dialysis and dried by lyophilization.

Into an amber vial, 4,5-bis(4-benzoylphenylmethyleneoxy) benzene-1,3-disulfonic acid (5 mg)(DBDS), prepared as described in U.S. Pat. No. 6,278,018 (Example 1) and commercially available from SurModics, Inc. (Eden Prairie, Minn.) was weighed and dissolved into deionized water at a concentration of 1 mg/mL. The solution was vortexed and sonicated to ensure a homogeneous mixture.

Poly(ethylene glycol) diacrylate (Sigma-Aldrich, St. Louis, Mo.; Avg. MW=700, cat. #455008) was weighed and dissolved into the DBDS solution at a concentration of 200 mg/mL for preparation of the DBDS/PEG-diacrylate solution.

A DBDS/photo-PA-PEG-AMPS was prepared by weighing and dissolving photo-PA-PEG-AMPS into a 1 mg/mL DBDS solution at a concentration of 60 mg/mL.

A solution was then prepared by mixing the DBDS/PEG-diacrylate solution with the DBDS/photo-PA-PEG-AMPS solution at a 1:1 ratio (v/v). Gel formation was carried out by placing the solution under ultraviolet light for five minutes to crosslink the polymeric components.

The concentrations and ratios of reagents can be altered to tune the physical properties of the formed gel.

### EXAMPLE 2

### Linear and Branched Poly(Ethylene Glycol) Diacrylates and Photo-PA-PEG-AMPS Crosslinkable Polymers for Gel Formation

A DBDS/PEG-diacrylate solution was prepared by weighing and dissolving the photoinitiator DBDS into deionized water at a concentration of 1 mg/mL in an amber vial. The solution was vortexed and sonicated to ensure a homogeneous mixture. Poly(ethylene glycol) diacrylate (Example 1) was weighed and dissolved into the DBDS initiator solution at a concentration of 200 mg/mL.

A DBDS/photo-PA-PEG-AMPS (Example 1) was prepared by weighing and dissolving photo-PA-PEG-AMPS into a 1 mg/mL DBDS solution at a concentration of 60 mg/mL.

A DBDS/PEG-triacrylate (trimethylolpropane ethoxylate (20/3 EO/OH) triacrylate macromer “PEG-triacrylate macromer” which is described in Example 5 of commonly assigned U.S. Patent Application Publication No. 2004/0202774A1 (Chudzik, et al.)) solution was prepared by weighing and dissolving PEG triacrylate into a 1 mg/mL DBDS solution at a concentration of 125 mg/mL.

A mixture was prepared by combining the DBDS/PEG-triacrylate, DBDS/PEG-diacrylate, and DBDS/photo-PA-PEG-AMPS solutions at a 1:10:10 ratio (v/v).

Gel formation was carried out by placing the solution under ultraviolet light for five minutes to crosslink the polymeric components.

The concentrations and ratios of reagents can be altered to tune the physical properties of the formed gel.

### EXAMPLE 3

### Poly(Ethylene Glycol) Diacrylates and Photo-PA Crosslinkable Polymers for Gel Formation

A photo-derivatized acrylamide monomer (BBA-APMA) was prepared by the reaction of N-(3-aminopropyl)methacrylamide (APMA) and benzoylbenzoyl chloride (BBA-Cl) followed by purification by recrystallization. The BBA-APMA monomer was then copolymerized with acrylamide in tetrahydrofuran (THF) resulting in formation of a white precipitate. The solid was filtered, dialyzed and lyophilized to give the final product. The resulting polymer was polyacrylamide-(3.5%)BBA-APMA-(0.4)BBA (photo-PA).

A DBDS/PEG-diacrylate (300 mg/mL) solution was prepared/

A DBDS/photo-PA solution was prepared by weighing and dissolving photo-PA into a 1 mg/mL DBDS solution at a concentration of 60 mg/mL.

A solution was then prepared by mixing the DBDS/PEG-diacrylate solution with the DBDS/photo-PA solution at a 1:1 ratio (v/v). Gel formation was carried out by placing the solution under ultraviolet light for five minutes to crosslink the polymeric components. The concentrations and ratios of reagents can be altered to tune the physical properties of the formed gel.

### EXAMPLE 4

### Poly(Ethylene Glycol) Diacrylate and Photo-PVP-APMA Crosslinkable Polymers for Gel Formation

A photo-derivatized vinyl pyrrolidone polymer (photo-PVP) was prepared by copolymerization of 1-vinyl-2-pyrrolidone and N-(3-aminopropyl)methacrylamide (“APMA”), followed by photoderivatization of the polymer using 4-benzoylbenzoyl chloride under Schotten-Baumann conditions as described in U.S. Pat. No. 5,414,075.

A DBDS/PEG-diacrylate solution was prepared as in Example 1.

A DBDS/photo-PVP solution was prepared by weighing and dissolving photo-PVP into a 1 mg/mL DBDS solution at a concentration of 60 mg/mL.

A solution was then prepared by mixing the DBDS/PEG-diacrylate solution with the DBDS/photo-PVP solution at a 1:1 ratio (v/v). Gel formation was carried out by placing the solution under ultraviolet light for five minutes to crosslink the polymeric components. The concentrations and ratios of reagents can be altered to tune the physical properties of the formed gel.

### Swelling Testing

Solutions as prepared in Examples 1-4 were pipetted into silicon tubing (HelixMark, Carpinteria, Calif.) with an inner diameter of 3.175 mm. To crosslink the material, the tubing was placed into a Dymax 2000-EC series UV chamber for five minutes. The filament was removed from the tubing as an opaque polymer. The filament was fully dried in a dry chamber for 18 hours (air drying). The diameter of the polymer filaments were measured using a Leica MZ12₅ stereomicroscope with Techniquip lighting and ImagePro-Plus software version 6.1. Next, the filament were placed into a glass vial with 1× phosphate buffer solution and hydrated at 37° C. The stereomicroscope was again used to measure the diameter of the filaments to determine swelling.

### Material Strength Testing

Solutions as prepared in Examples 1-4 were injected into a 9 mm wide×4 mm deep diameter Teflon well, A Dymax 2000-EC series UV flood lamp was used to initiate cross-linking of the polymers. The samples were placed in the chamber 20 cm from the light source for five minutes to ensure a complete photochemical reaction.

The physical properties of the gels were determined by compression force testing. A TAXT2 texture analyzer with 5 mm diameter ball probe was used to determined compression strength. The procedure used a test speed of 0.5 mm/sec and a trigger force of 4 grams. The probe compressed to 25% of the depth of the material as compared to the calibration depth.

TABLE 2
Compression and swelling data for referenced examples.

| | | | Swelling - size |
|---|---|---|---|
| Example | Reagents | Force (g) | increase % |
| Control | Poly(ethylene glycol) diacrylate^{a} | N/A | N/A |
| 1 | Poly(ethylene glycol) diacrylate/Photo-PA-PEG-AMPS | 29.682 | 148% |
| 2 | Poly(ethylene glycol) diacrylate/Photo-PA-PEG-AMPS/ | 58.845 | 141% |
| | branched PEG-triacrylate | | |
| 3 | Poly(ethylene glycol) diacrylate/Photo-PA-AMPS | 22.85 | 80% |
| 4 | Poly(ethylene glycol) diacrylate/Photo-PVP-APMA | 43.556 | 106% |

^{a}The PEGDA solution did not polymerize to a gel when used at a concentration of 200 mg/mL.

What is claimed is:

1. A medical implant comprising:
a biocompatible swellable or swollen polymeric matrix comprising first and second polymer-containing segments covalently linked via reacted groups, wherein the first polymer-containing segment comprises a hydrophilic polymer comprising pendent polymerized groups, and the second polymer-containing segment comprises a hydrophilic polymer comprising pendent reacted photogroups, and
a non-porous article having a surface which the biocompatible swellable or swollen polymeric matrix is associated with.

2. The medical implant of claim 1 wherein the first polymer-containing segment comprises two pendent polymerized groups.

3. The medical implant of claim 1 wherein the first polymer-containing segment comprises an oxyalkylene polymer.

4. The medical implant of claim 1 wherein the first polymer-containing segment has a molecular weight in the range of 250 Da to 20 kDa.

5. The medical implant of claim 1 wherein the first polymer-containing segment having a linear structure comprises a polymer selected from the group consisting of poly(ethylene oxide) (PEO), poly(ethyloxazoline), poly(propylene oxide) (PPO), poly(ethylene glycol) (PEG), copolymers of polyethylene glycol and polypropylene oxide (PEG-PPO), and polyvinyl alcohol.

6. The medical implant of claim 1 wherein the first polymer-containing segment has a branched structure, or the polymeric matrix further comprises a third polymer-containing segment having a branched structure.

7. The medical implant of claim 6 wherein the branched structure comprises poly(ethylene glycol) portions.

8. The medical implant of claim 6 wherein the branched structure is selected from the group consisting of: 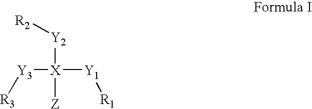
wherein X is C or S, or a homo- or heterocyclic ring; Y₁, Y₂, and Y₃ are independently, —Cₙ—O—, wherein n is 0 or an integer of 1 or greater; R₁, R₂, and R₃, are independently hydrophilic polymeric portions, which can be the same or different, and R₁, R₂, and R₃ independently have one or more pendent polymerized group(s); and Z is a non-polymeric group; 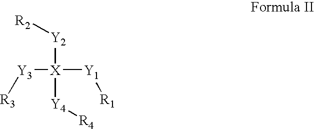
wherein X is C or S, or a homo- or heterocyclic ring; Y₁, Y₂, Y₃, and Y₄ are independently, —Cₙ—O—, wherein n is 0 or an integer of 1 or greater; R₁, R₂, R₃, and R₄, are independently hydrophilic polymeric portions, which can be the same or different, and R₁, R₂, R₃, and R₄ independently have one or more pendent polymerized group(s); and 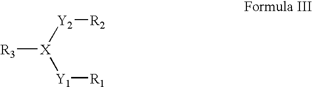
wherein X is selected from N, C—H, or S—H, or a homo- or heterocyclic ring; Y₁ and Y₂ are —Cₙ—O—, wherein n is 0 or an integer of 1 or greater; and R₁, R₂, and R₃, are independently hydrophilic polymeric portions, which can be the same or different, and R₁, R₂, and R₃ independently have one or more pendent polymerized group(s).

9. The medical implant of claim 1 wherein the second polymer-containing segment comprises non-oxyalkylene monomeric units.

10. The medical implant of claim 1 wherein the second polymer-containing segment comprises monomeric units selected from (meth)acrylamide, vinyl pyrrolidone, and (meth)acrylic acid.

11. The medical implant of claim 9 wherein the second polymer-containing segment comprises copolymer comprising oxyalkylene and non-oxyalkylene monomeric units.

12. The medical implant of claim 1 wherein the second polymer-containing segment comprises amphiphilic monomeric units selected from the group consisting of diacetone acrylamide (DAA), vinyloxyethanol (VOE), 2-acrylamido-2-methylpropane (AMPS), and methyl acryloyl lactate (ALM).

13. The medical implant of claim 1 wherein the molecular weight of the first polymer-containing segment is less than the second polymer-containing segment.

14. The medical implant of claim 1 wherein the first polymer-containing segment and the second polymer-containing segment are present in the matrix at a weight ratio in the range of 9:1 to 1:3, respectively.

15. The medical implant of claim 14 wherein the first polymer-containing segment and the second polymer-containing segment are present in the matrix at a weight ratio in the range of 4:1 to 1:2, respectively.

16. The medical implant of claim 1, wherein the biocompatible swellable or swollen polymeric matrix is in the form of an overmold on the non-porous article.

17. The medical implant of claim 1 comprising a radiopaque agent.

18. A method for occluding a target site in the body comprising steps of:
placing at a target site in the body a biocompatible swellable matrix comprising first and second polymer-containing segments covalently linked via reacted groups, wherein the first polymer-containing segment comprises a hydrophilic polymer comprising pendent polymerized groups, and second polymer-containing segment comprises a hydrophilic polymer comprising pendent reacted photogroups; and
allowing the matrix to swell at the target site, which occludes the target site.

19. The method of claim 18, wherein the matrix swells at the target site to a weight of 1.5 times or greater a weight of the matrix in a dehydrated form.

20. The method of claim 18, wherein the matrix exerts a swelling force of 100 g/cm² or greater upon swelling.

21. The method of claim 18, wherein the matrix swells to a size that is at least 25% greater than its size in a dehydrated form.
